(19) United States
(12) Patent Application Publication
(10) Pub. No.: US 2009/0203786 A1
(43) Pub. Date: Aug. 13, 2009

(54) Methods and Composition for Improving Cognitive Function

(76) Inventors: Mark K. Waldron, Savigny (CH); Arleigh J. Reynolds, Salcha, AK (US)

Correspondence Address: Wendell Ray Guffey;Nestle Purina Petcare Global Resources, Checkerboard Square 11T, St Louis, MO, 63164, US

(21) Appl. No.: 11/992,280

(22) PCT Filed: Oct. 2, 2006
(86) PCT No.: PCT/US06/38287

§ 371 Date: Mar. 26, 2009

Related U.S. Application Data
(60) Provisional application No. 60/722,788, filed on Sep. 30, 2005.

(51) Int. Cl. A61K 31/202; A61K 31/201

(52) U.S. Cl. 514/560

(57) ABSTRACT

Compositions and methods for enhancing cognitive function in animals are disclosed. The compositions and methods utilize long chain polyunsaturated fatty acids.

### CROSS-REFERENCE TO RELATED APPLICATIONS

This application claims priority to U.S. Provisional Application Ser. No. 60/722,788 filed Sep. 30, 2005, and to PCT Application No. PCT/US2006/038287 filed on Oct. 2, 2006, the disclosures of which are incorporated herein by reference.

### FIELD OF THE INVENTION

The present invention is related to mammalian nutrition and effects thereof on cognitive function. In particular, the present invention utilizes long chain polyunsaturated fatty acids, administered during gestation through the maternal diet, or post-parturition from maternal milk or directly through diet as the animal matures, to improve problem solving, memory retention, and mental stability.

### BACKGROUND OF THE INVENTION

Various publications, including patents, published applications, technical articles and scholarly articles are cited throughout the specification. Each of these cited publications is incorporated by reference herein, in its entirety. Full citations for publications not cited fully within the specification are set forth at the end of the specification.

Both (n-3) and (n-6) classes of long-chain polyunsaturated fatty acids (LCPUFA) are important in perinatal development. Increasing evidence indicates that the (n-3) fatty acids are of particular importance in development of the central nervous system (CNS). In primates, neural development begins in the third trimester of gestation, peaks about the time of birth, and continues for about 18-24 months after parturition (Menard, C R et al. 1998, Martinez, M 1992). Although differences are likely, it is believed that this pattern of development holds true among most mammalian species. (Bauer J E et al. 2004).

During this developmental period, fatty acids such as arachidonic acid (AA) and docosahexaenoic acid (DHA) are rapidly incorporated into the neural tissues (Sinclair, AJ 1975, Greiner, R C et al. 1997). Accumulation of DHA occurs primarily during late gestation and in the postnatal period of development, although enrichment of DHA into neurological tissues continues post parturition (Carnielli, V P et al. 1998). DHA is primarily found in the serine and ethanolamine phospholipids in retinal and neurological tissue.

The incorporation of supplemental DHA into neurological tissue has been investigated. In vitro studies showed that rat retina neuronal cells incubated with DHA had four- to six-fold more DHA than cells incubated with other fatty acids (Rotstein, N P et al. 1999). The addition of other fatty acids in that study had no effect on altering cell membrane fatty acid compositions. The report suggested that retinal neurons have specific mechanisms for handling fatty acids of different length and desaturation and the selective uptake DHA. Indeed, there appears to be at least one mechanism by which DHA is selectively taken up by neural and retinal tissues. Studies in pigs showed that diets supplemented with DHA increased brain accumulation of DHA during the postnatal growth period (Morris S A et al. 1999). In addition, in vivo studies have shown that supplemental DHA is accumulated into neurological tissues in piglets, kittens, and non-human primates (Pawlosky, R J et al. 1997, Green, P et al. 1996). Conversely, a deficiency of DHA has been shown to be deleterious in laboratory species. For example, rats fed deficient diets had decreased memory and cognitive ability. (Moriguichi T et al. 2000). Similar results have been observed in preterm human infants and in Rhesus monkeys fed DHA-deficient diets (Carlson S E et al. 1993; and Neuringer M et al. 1984).

The high amounts of DHA found in the brain and in the retina suggest a functional role in those tissues (Litman, B J et al. 2001). In non-human primates and human infants, supplemental DHA has been shown to increase visual acuity and cognitive abilities (Willats P 2002; Uauy R et al. 2003; Gil A et al. 2003). Deficiency of (n-3) polyunsaturated fatty acids during the developmental phase of neural tissues can result in irreversible functional abnormalities.

Dietary supplementation with DHA and AA has also been shown to improve learning in rats and rhesus monkeys. (Lothaller M A et al. (1991), Greiner R S et al. (1999), and Wainwright P E et al. (1999)). In addition, children who were fed formulas supplemented with these LCPUFAs also showed improved visual acuity and higher scores on a mental development index test (MDI) than a matched cohort fed the identical formula devoid of DHA and AA. (Birch E E et al. (2000)).

Not all studies investigating the effects of DHA and AA supplementation upon central nervous system development have shown such positive results. (Gibson R A et al. 1997)). A closer examination of the amounts of AA and DHA fed, as well as the period of development of the animal, may account for the differences between those studies which showed a benefit of supplementation and those which did not. There appears to be a window of time during early development where LCPUFA supplementation is most beneficial. This time may vary from species to species, depending upon when CNS growth and development is most rapid. (Connor W E et al. (1990), and (Liu C C et al. (1987)). For example, Rhesus monkeys showed a significant increase in the DHA content of their cerebral cortex after one week of supplementation. Cerebral DHA concentrations continued to increase for 12 weeks, at which point they stabilized at 7 times the pre-supplementation value. (Connor W E et al. (1990)). It thus appears that supplementation must take place during a time when the brain will incorporate DHA and AA at the maximal rate and concentration, and must continue for a long enough period to allow saturation of the plasma membranes in the neurological tissue. Although maximal incorporation of LCPUFA may take place during a limited window of time, adequate intakes of the LCPUFA may be required throughout life, as evidenced by the fact that the half-life of DHA in Rhesus monkey brain appears to be only 21 days. (Connor W E et al. (1990)).

As important as the timing and duration of supplementation is the amount of each type of LCPUFA provided in the animal's diet. One study showed that children receiving only DHA supplementation experienced a significant increase in the concentration of DHA in their red blood cell membranes, although no significant change in their MDI score was observed relative to non-supplemented children. (Gibson R A et al. (1997)). Studies in children (Carlson S E (1996)), rats (Greiner R S et al. (1999)), and rhesus monkeys (Connor W E et al. (1990)), have shown that DHA supplementation in the absence of AA supplementation leads to an increase in CNS and RBC concentrations of DHA with a concomitant decrease in the AA concentration of these membranes. Most studies which have recognized a benefit of DHA supplementation have supplemented AA at the same time.

Despite the knowledge regarding the benefits of DHA and AA, and the benefits of dietary supplementation of DHA and AA in humans and certain laboratory mammals, the benefits of DHA and AA in the neurological development of domestic and companion animals such as dogs and cats remains largely unexplored. Thus, there is a need in the art to provide compositions and methods to impart the benefits of DHA and AA, and LCPUFA generally, to these types of animals, to improve their cognitive function and to provide related neurological advantages. Enriching reproduction/lactation and growth diets with LCPUFA can provide animals with superior cognitive function that translates into a more satisfactory pet-owner bond. The present invention meets this need.

### SUMMARY OF THE INVENTION

One aspect of the invention features composition comprising one or more long chain polyunsaturated fatty acids (LCPUFA), in an amount effective for improving cognitive function in an animal. In various embodiments, the composition is a pet food composition or a dietary supplement. In various embodiments, the animal is a companion animal, preferably a dog or cat. The LCPUFA may include at least one of arachidonic acid, eicosapentaenoic acid, docosapentaenoic acid, or docosahexaenoic acid, and may be present in an amount of at least about 0.1% to about 10% by weight of the composition, more specifically between about 0.4 to about 5.0% by weight of the composition, and even more specifically, between about 2% and about 2.5% by weight of the composition.

Another aspect of the invention features a method for enhancing cognitive function in an animal comprising administering to the animal one or more LCPUFA in an amount effective to enhance cognitive function in the animal. In this aspect of the invention, the LCPUFA may include one or more of arachidonic acid, eicosapentaenoic acid, docosapentaenoic acid, or docosahexaenoic acid. In certain embodiments, the animal is a companion animal, preferably a dog or a cat.

In one embodiment, the LCPUFA are administered to the animal during gestation. In another embodiment, the LCPUFA are administered to the animal during the period spanning parturition through about twelve weeks after parturition. In another embodiment, the LCPUFA are administered to the animal during gestation and during the period spanning parturition through about twelve weeks after parturition.

In various embodiments, the LCPUFA are administered in a pet food composition or a dietary supplement. In another embodiment, the LCPUFA are administered in milk from a lactating animal to which has been administered one or more LCPUFA. In other embodiments, the LCPUFA are administered in a pet food composition or dietary supplement and in milk from a lactating animal to which has been administered one or more LCPUFA.

The LCPUFA may be administered to the animal in various regimens. In one embodiment, the LCPUFA are administered on a daily basis. In another embodiment, the LCPUFA are administered to the animal as part of a dietary regimen. In specific embodiments, the duration of the dietary regimen ranges from parturition to about 12 weeks of age.

Other features and advantages of the invention will be understood from the detailed description and examples that follow.

### DETAILED DESCRIPTION OF ILLUSTRATIVE EMBODIMENTS

Proper neural development of mammalian species depends on the presence of LCPUFA, especially DHA, during fetal development and the perinatal period. DHA and AA are of particular importance in this regard because they have been demonstrated to enhance cognitive abilities in certain human and non-human primates and in laboratory animals. In accordance with the present invention, it has been demonstrated that long chain polyunsaturated fatty acids made available to animals pre-natally through maternal diet and post-natally through the animals' diet is effective in promoting enhanced cognitive abilities in the animals. Enhanced cognitive function is achieved when LCPUFA are administered to the animals indirectly through their mother during gestation, directly to the animals through their diet, or administered to the animals in combinations thereof.

It is thus important to ensure that LCPUFA such as DHA and AA are in plentiful supply in the blood of the female mammal during gestation, and are in plentiful supply in the blood of the neonatal animal through the perinatal period, and through development of the young animal. One means to accomplish this goal is through the diet of both the pregnant female and her developing newborns.

Of particular note in this regard is that dietary LCPUFA can be provided to the newborn animal through the milk of the lactating female. In humans, dietary supplementation with fishmeal or fish oil supplements results in the deposition of (n-3) fatty acids, especially DHA, into the breast milk. The DHA content of human breast milk is proportional to the DHA content of the maternal diet. This observation appears to hold true for other mammals, including non-human primates, rats, and dogs. A dose effect is observed between the DHA content of the diet and the DHA content of the milk of lactating female dogs. (Bauer J E et al. 2004 abstract). Thus, one means to provide dietary LCPUFA to neonatal and young animals, particularly during the perinatal period, is through the milk of the lactating female.

### DEFINITIONS

Various terms relating to the methods and other aspects of the present invention are used throughout the specification and claims. Such terms are to be given their ordinary meaning in the art unless otherwise indicated. Other specifically defined terms are to be construed in a manner consistent with the definition provided herein.

The following abbreviations may be used in the specification and examples: AA, arachidonic acid; ANOVA, analysis of variance; BW, body weight; DHA, docosahexaenoic acid; DM, dry matter; DPA, docosapentaenoic acid; EPA, eicosapentaenoic acid; LCPUFA, long chain polyunsaturated fatty acids.

“Effective amount” refers to an amount of a compound, material, or composition, as described herein that is effective to achieve a particular biological result. Such results include, but are not limited to, enhancing cognitive function, improving problem solving abilities, improving memory, and improving mental stability. Such effective activity may be achieved, for example, by administering the compositions of the present invention to the animal.

The term “cognitive function” refers to the special, normal, or proper physiologic activity of the brain, including, without limitation, mental stability, memory/recall abilities, problem solving abilities, reasoning abilities, thinking abilities, judging abilities, capacity for learning, perception, intuition, and awareness. “Enhanced cognitive function” refers to any improvement in the special, normal, or proper physiologic activity of the brain, including, without limitation, mental stability, memory/recall abilities, problem solving abilities, reasoning abilities, thinking abilities, judging abilities, capacity for learning, perception, intuition, and awareness, as measured by any means suitable in the art.

As used herein, “long chain polyunsaturated fatty acids” or “LCPUFA” refers to any monocarboxylic acid having at least 20 carbon atoms and at least two double bonds. Non-limiting examples of LCPUFA include (n-6) fatty acids such as arachidonic acid, and (n-3) fatty acids such as eicosapentaenoic acid, docosapentaenoic acid and docosahexaenoic acid.

The present invention relates to any animal, preferably a mammal, and more preferably, companion animals. A “companion animal” is any domesticated animal, and includes, without limitation, cats, dogs, rabbits, guinea pigs, ferrets, hamsters, mice, gerbils, horses, cows, goats, sheep, donkeys, pigs, and the like. Dogs and cats are most preferred, and dogs are exemplified herein.

As used herein, the term “pet food” or “pet food composition” means a composition that is intended for ingestion by an animal, and preferably by companion animals. A “complete and nutritionally balanced pet food,” is one that contains all known required nutrients in appropriate amounts and proportions based on recommendations of recognized authorities in the field of companion animal nutrition, and is therefore capable of serving as a sole source of dietary intake to maintain life or promote production, without the addition of supplemental nutritional sources. Nutritionally balanced pet food compositions are widely known and widely used in the art.

As used herein, a “dietary supplement” is a product that is intended to be ingested in addition to the normal diet of an animal.

### Compositions

One embodiment of the invention features compositions comprising one or more LCPUFA in an amount effective for the enhancement of cognitive function in animals. The LCPUFA can be present in the composition as an ingredient or additive. In one preferred embodiment, the composition comprises (n-3) fatty acids such as EPA, DPA and, most preferably, DHA. In another preferred embodiment, the composition comprises (n-6) fatty acids such as AA. In more preferred embodiment, the composition comprises combinations of (n-3) and (n-6) fatty acids, most preferably DHA and AA. The compositions enrich the blood plasma with LCPUFA in animals to which the composition is administered, and enrich the milk of a lactating animal with LCPUFA in lactating animals to which the composition is administered.

In a preferred embodiment, the compositions of the invention are pet food compositions. These will advantageously include foods intended to supply necessary dietary requirements, as well as treats (e.g., biscuits) or other dietary supplements. Optionally, the pet food compositions can be a dry composition (for example, kibble), semi-moist composition, wet composition, or any mixture thereof. In another preferred embodiment, the composition is a dietary supplement, such as a gravy, drinking water, beverage, yogurt, powder, granule, paste, suspension, chew, morsel, treat, snack, pellet, pill, capsule, tablet, or any other delivery form. In a detailed embodiment, the dietary supplement can comprise a high concentration of LCPUFAs or DHA and AA such that the supplement can be administered to the animal in small amounts, or in the alternative, can be diluted before administration to an animal. The dietary supplement may require admixing with water prior to administration to the animal.

The composition may be refrigerated or frozen. The LCPUFA may be pre-blended with the other components of the composition to provide the beneficial amounts needed, may be coated onto a pet food composition, or may be added to the composition prior to offering it to the animal, for example, using a sprinkled powder or a mix.

The compositions of the invention comprise LCPUFA in an amount effective to enhance cognitive function in an animal to which the composition has been administered. For pet foods, the amount of (n-3) LCPUFA as a percentage of the composition is in the range of about 0.1% to about 10% in certain embodiments, up to 5% in other embodiments, and about 2.0% in specific embodiments, of the composition on a dry matter basis, although a greater percentage can be supplied. In various embodiments, the amount is about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or more of the composition on a dry matter basis. The amount of (n-6) LCPUFA as a percentage of the composition is in the range of about 0.1% to about 10% in certain embodiments, up to 5% in other embodiments, and about 2.0% in specific embodiments, of the composition on a dry matter basis, although a greater percentage can be supplied. In various embodiments, the amount is about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9%, 5.0%, or more of the composition on a dry matter basis. Dietary supplements may be formulated to contain several-fold higher concentrations of LCPUFA, to be amenable for administration to an animal in the form of a tablet, capsule, liquid concentrated, or other similar dosage form, or to be diluted before administrations, such as by dilution in water, spraying or sprinkling onto a pet food, and other similar modes of administration.

In another embodiment, the amount of LCPUFA in the composition is a function of an amount required to establish a specified concentration of LCPUFA in the blood serum of the animal. The specified concentration of LCPUFA in the blood serum is in the range of about 0.1% to about 25% of total fatty acid content in the blood serum. In still another embodiment, the amount of LCPUFA in the composition is a function of an amount required to establish a specified concentration of LCPUFA in the milk of the lactating animal. The specified concentration of (n-3) LCPUFA in the milk is in the range of about 0.1% to about 7.0% of total fatty acid content in the milk. The specified concentration of (n-6) LCPUFA in the milk is in the range of about 0.1% to about 7.0% of total fatty acid content in the milk.

The sources of each of the LCPUFA can be any suitable source, synthetic or natural. Preferred sources of LCPUFA include, without limitation, cyanobacteria and algae, such as Ciypthecodinium cohnii and Schizochytrium spp., and fish, especially cold-water fish such as salmon, tuna, mackerel, herring, sea bass, striped bass, shark, halibut, catfish, sardines, shrimp, and clams, and their extracted oils, or the LCPUFA may be synthesized de novo according to any means suitable in the art.

The compositions of the invention can optionally comprise supplementary substances such as minerals, vitamins, salts, condiments, colorants, and preservatives. Non-limiting examples of supplementary minerals include calcium, phosphorous, potassium, sodium, iron, chloride, boron, copper, zinc, manganese, iodine, selenium and the like. Non-limiting examples of supplementary vitamins include vitamin A, various B vitamins, vitamin C, vitamin D, vitamin E, and vitamin K. Additional dietary supplements may also be included, for example, niacin, pantothenic acid, inulin, folic acid, biotin, amino acids, and the like.

The compositions of the invention can optionally comprise one or more supplementary substances that promote or sustain general neurologic health, or further enhance cognitive function. Such substances include, without limitation, choline, phosphatidylserine, acetyl-L-carnitine, and herbal extracts such as Ginko biloba, Bacopa monniera, Convolvulus pliiricaulis, and Leucojum aestivum.

In various embodiments, pet food or pet treat compositions of the invention can comprise, on a dry matter basis, from about 15% to about 50% crude protein, by weight of the composition. The crude protein material may comprise vegetable proteins such as soybean, cottonseed, and peanut, or animal proteins such as casein, albumin, and meat protein. Non-limiting examples of meat protein useful herein include pork, lamb, equine, poultry, fish, and mixtures thereof.

The compositions may further comprise, on a dry matter basis, from about 5% to about 40% fat, by weight of the composition. The compositions may further comprise a source of carbohydrate. The compositions may comprise, on a dry matter basis, from about 15% to about 60% carbohydrate, by weight of the composition. Non-limiting examples of such carbohydrates include grains or cereals such as rice, corn, milo, sorghum, alfalfa, barley, soybeans, canola, oats, wheat, and mixtures thereof. The compositions may also optionally comprise other materials such as dried whey and other dairy by-products.

The compositions may also comprise at least one fiber source. A variety of soluble or insoluble fibers may be utilized, as will be known to those of ordinary skill in the art. The fiber source can be beet pulp (from sugar beet), gum arabic, gum talha, psyllium, rice bran, carob bean gum, citrus pulp, pectin, fructooligosaccharide additional to the short chain oligofructose, mannanoligofructose, soy fiber, arabinogalactan, galactooligosaccharide, arabinoxylan, or mixtures thereof. Alternatively, the fiber source can be a fermentable fiber. Fermentable fiber has previously been described to provide a benefit to the immune system of a companion animal. Fermentable fiber or other compositions known to those of skill in the art which provide a prebiotic composition to enhance the growth of probiotic microorganisms within the intestine may also be incorporated into the composition to aid in the enhancement of the benefit provided by the present invention to the immune system of an animal. Additionally, probiotic microorganisms, such as Lactobacillus or Bifidobacterium species, for example, may be added to the composition.

In a detailed embodiment, the composition is a complete and nutritionally balanced pet food. In this context, the pet food may be a wet food, a dry food, or a food of intermediate moisture content, as would be recognized by those skilled in the art of pet food formulation and manufacturing. “Wet food” describes pet food that is typically sold in cans or foil bags, and has a moisture content typically in the range of about 70% to about 90%. “Dry food” describes pet food which is of a similar composition to wet food, but contains a limited moisture content, typically in the range of about 5% to about 15%, and therefore is presented, for example, as small biscuit-like kibbles. The compositions and dietary supplements may be specially formulated for adult animals, or for older or young animals, for example, a “puppy chow,” “kitten chow,” or “senior” formulation. In general, specialized formulations will comprise energy and nutritional requirements appropriate for animals at different stages of development or age.

Certain aspects of the invention are preferably used in combination with a complete and balanced food (for example, as described in National Research Council, 1985, Nutritional Requirements for Dogs, National Academy Press, Washington D.C., or Association of American Feed Control Officials, Official Publication 1996). That is, compositions comprising LCPUFA, or DHA and AA according to certain aspects of this invention are preferably used with a high-quality commercial food. As used herein, “high-quality commercial food” refers to a diet manufactured to produce the digestibility of the key nutrients of 80% or more, as set forth in, for example, the recommendations of the National Research Council above for dogs, or in the guidelines set forth by the Association of American Feed Control Officials. Similar high nutrient standards would be used for other animals.

The skilled artisan will understand how to determine the appropriate amount of LCPUFA or DHA and AA to be added to a given composition. Such factors that may be taken into account include the type of composition (e.g., pet food composition versus dietary supplement), the average consumption of specific types of compositions by different animals, and the manufacturing conditions under which the composition is prepared. Preferably, the concentrations of LCPUFA or DHA and AA to be added to the composition are calculated on the basis of the energy and nutrient requirements of the animal. According to certain aspects of the invention, the LCPUFA or DHA and AA can be added at any time during the manufacture and/or processing of the composition. This includes, without limitation, as part of the formulation of the pet food composition or dietary supplement, or as a coating applied to the pet food composition or dietary supplement.

The compositions can be made according to any method suitable in the art such as, for example, that described in Waltham Book of Dog and Cat Nutrition, Ed. ATB Edney, Chapter by A. Rainbird, entitled “A Balanced Diet” in pages 57 to 74, Pergamon Press Oxford.

### Methods

Another aspect of the invention features methods for enhancing the cognitive function in an animal comprising administering to the animal a composition comprising one or more LCPUFA in an amount effective to enhance cognitive function in the animal. In a detailed embodiment, the composition is a pet food composition or a dietary supplement, as exemplified herein. In a further detailed embodiment, the LCPUFA is an (n-3) LCPUFA, including but not limited to, EPA, DPA and DHA. In another detailed embodiment, the LCPUFA is an (n-6) LCPUFA, including but not limited to, AA. In a still another detailed embodiment, the LCPUFA is a combination of (n-3) and (n-6) LCPUFA, including but not limited to, EPA, DPA, DHA, and AA. Animals may include any domesticated or companion animals as described above. In certain embodiments, the animal is a companion animal such as a dog or cat. In one embodiment, the animal is a dog.

The compositions can be administered to the animal by any of a variety of alternative routes of administration. Such routes include, without limitation, oral, intranasal, intravenous, intramuscular, intragastric, transpyloric, subcutaneous, rectal, and the like. Preferably, the compositions are administered orally. As used herein, the term “oral administration” or “orally administering” means that the animal ingests or a human is directed to feed, or does feed, the animal one or more of the inventive compositions described herein.

Wherein the human is directed to feed the composition, such direction may be that which instructs and/or informs the human that use of the composition may and/or will provide the referenced benefit, for example, the enhancement of cognitive function in the animal. Such direction may be oral direction (e.g., through oral instruction from, for example, a physician, veterinarian, or other health professional, or radio or television media (i.e., advertisement), or written direction (e.g., through written direction from, for example, a physician, veterinarian, or other health professional (e.g., prescriptions), sales professional or organization (e.g., through, for example, marketing brochures, pamphlets, or other instructive paraphernalia), written media (e.g., internet, electronic mail, or other computer-related media), and/or packaging associated with the composition (e.g., a label present on a container holding the composition).

Administration can be on an as-needed or as-desired basis, for example, once-monthly, once-weekly, daily, or more than once daily. Similarly, administration can be every other day, week, or month, every third day, week, or month, every fourth day, week, or month, and the like. Administration can be multiple times per day. When utilized as a supplement to ordinary dietetic requirements, the composition may be administered directly to the animal or otherwise contacted with or admixed with daily feed or food. When utilized as a daily feed or food, administration will be well known to those of ordinary skill.

Administration can also be carried out as part of a diet regimen in the animal. For example, a diet regimen may comprise causing the regular ingestion by the animal of a composition comprising one or more LCPUFA, preferably DHA and AA, in an amount effective to enhance cognitive function in the animal. Regular ingestion can be once a day, or two, three, four, or more times per day, on a daily basis. The goal of regular ingestion is to provide the animal with the preferred daily dose of LCPUFA, as exemplified herein.

The daily dose of LCPUFA can be measured in terms of grams of LCPUFA per kg of body weight (BW) of the animal or in terms of a percentage of total daily caloric requirement of the animal. The daily dose of LCPUFA can range from about 0.01 g/kg to about 2.0 g/kg BW of the animal. Preferably, the daily dose of LCPUFA is from about 0.1 g/kg to about 1.25 g/kg BW of the animal. In an exemplary embodiment, the daily dose of LCPUFA consisted of 0.11 g/kg body weight of DHA and 0.056 g/kg body weight of AA.

In the alternative, the daily dose of LCPUFA can range from about 0.1 to about 15% of the total daily caloric requirement of the animal. Preferably, the daily dose of LCPUFA is from about 3% to about 8% of the total daily caloric requirement of the animal. More preferably, the daily dose of LCPUFA is from about 6 to about 8% of the total daily caloric requirement of the animal.

According to the methods of the invention, administration of the LCPUFA, including administration as part of a diet regimen, can span a period of time ranging from gestation through the adult life of the animal. In a preferred embodiment, the duration of the administration ranges from gestation through about 36 months after parturition. In a more preferred embodiment, the duration of the administration ranges from gestation through about 30 months after parturition. In a still more preferred embodiment, the duration of the administration ranges from gestation through about 24 months after parturition. In a still more preferred embodiment, the duration of the administration ranges from gestation through about 18 months after parturition. In a still more preferred embodiment, the duration of the administration ranges from gestation through about 12 months after parturition. In a still more preferred embodiment, the duration of the administration ranges from gestation through about 40 weeks after parturition. In a still more preferred embodiment, the duration of the administration ranges from gestation through about 35 weeks after parturition. In a still more preferred embodiment, the duration of the administration ranges from gestation through about 30 weeks after parturition. In a still more preferred embodiment, the duration of the administration ranges from gestation through about 25 weeks after parturition. In a still more preferred embodiment, the duration of the administration ranges from gestation through about 20 weeks after parturition. In a still more preferred embodiment, the duration of the administration ranges from gestation through about 18 weeks after parturition. In a still more preferred embodiment, the duration of the administration ranges from gestation through about 16 weeks after parturition. In a still more preferred embodiment, the duration of the administration ranges from gestation through about 14 weeks after parturition. In a still more preferred embodiment, the duration of the administration ranges from gestation through about 12 weeks after parturition. In an alternative embodiment, the duration of the administration ranges from gestation through about 10 weeks after parturition. In another alternative embodiment, the duration of the administration ranges from gestation through about 8 weeks after parturition. In another alternative embodiment, the duration of the administration ranges from gestation through about 6 weeks after parturition.

In another embodiment, the inventive method comprises administering to the animal milk from a lactating animal to which has been administered one or more LCPUFA. The LCPUFA-enriched milk can be administered to an animal in order to enhance the cognitive function in that animal.

The LCPUFA can be administered to the lactating animal according to the inventive methods described herein. In a preferred embodiment, the lactating animal is administered a composition comprising one or more LCPUFA. In a more preferred embodiment, the lactating animal is administered a composition comprising DHA and AA. The composition comprising one or more LCPUFA that is administered to the lactating animal can be a pet food composition or dietary supplement, as exemplified herein. The composition may be administered to the lactating animal before conception, during gestation, and after parturition during the suckling period. The lactating animal may be the parent of the animal to which the milk is administered. The milk may be administered via suckling, or may be administered after isolation from the lactating animal. The milk can be administered on an as-needed or as-desired basis, or as part of a diet regimen, as described herein.

In another embodiment, the inventive method comprises administering LCPUFA to the animal during gestation, by passage from the mother animal to which has been administered one or more LCPUFA. In a preferred embodiment, the mother animal is administered a composition comprising one or more LCPUFA. In a more preferred embodiment, the mother animal is administered a composition comprising DHA and AA. The composition comprising one or more LCPUFA that is administered to the mother animal can be a pet food composition or dietary supplement, as exemplified herein. The composition may be administered to the mother animal from before the time of estrus through parturition.

In still another embodiment, the LCPUFA is administered to the animal both during gestation and after parturition according to the details set forth above.

The amount of composition utilized in the various embodiments of the methods of the invention may be dependent on a variety of factors, including the health, condition, and/or age of the animal, the quality of the pet food composition or dietary supplement, and species, size or breed of the animal.

Determination of the improvement of cognitive functions such as problem solving, memory, and mental stability of the animals achieved by practicing the methods of the invention may be determined by any means suitable in the art. Examples of suitable means are set forth in the examples that follow.

The following examples are provided to describe the invention in greater detail. The examples are intended illustrate, not to limit, the invention.

### Example 1

### Effect of Dietary Supplementation with LCPUFA on Cognitive Performance in Puppies

Animals and diets. The dogs were Husky-Pointer crossbreeds. Female dogs were maintained in indoor-outdoor kennels from breeding to 3 weeks post whelping. At this time each female and her litter was moved to a 4 by 5 meter pen with a large house. Pups were kept with their mothers until 10 weeks of age and were group housed in their pen until the end of the study. From birth onward, all pups were handled for 20-45 minutes 1-2 times a day. From 4 weeks of age until the end of the study, each litter was walked ½ to 1 mile daily, as a group.

Five pregnant females were fed Purina ProPlan Performance chicken and rice diet (Nestle-Purina Pet Care Co., St. Louis, Mo.) during gestation and lactation. Animals were fed to maintain an optimal body condition score (5/10) during gestation and lactation. Food was offered twice a day. Puppies from 5 litters were split as evenly as possible in terms of sex, and assigned to one of two dietary treatment groups: “A” (corn oil placebo), and “B” (DHA and AA supplementation). A total of 20 puppies were assigned to each group. Puppies were offered soaked basal diet twice a day beginning at 3 weeks of age. Supplements were administered as a percentage of dietary fat intake (2% for DHA, 1% for AA, and 3% for corn oil) once daily with the morning feeding. Group B received daily supplements of DHA and AA at 1% and 2% of total fatty acid content of their basal diet (Purina Pro Plan Performance chicken and rice diet). Puppies were weighed weekly, the weights recorded, and the dosage of supplements adjusted accordingly. They were handled and taken for walks to ensure proper socialization.

Cognitive function testing. Behavioral testing began at 8 weeks of age with the cry and shriek test. In this test, mental stability is evaluated by isolating the puppy in a cage and measuring how long it takes for it to cry once and then shriek or cry 3 times in succession. In the cry and shriek test, there was a numerical trend for the supplemented dogs to score better (take longer to cry or shriek), suggesting better mental stability. These data are summarized in Table 1.

TABLE 1
Mean times (seconds) for Cry and Shriek Test

| | Cry time | Shriek Time |
|---|---|---|
| Treatment A (corn oil) | 18.5 | 35 |
| Treatment B (DHA and AA) | 32 | 51 |
| p-value control = treatment | 0.41 | 0.17 |

At 10 weeks of age, the puppies' problem solving abilities were evaluated by means of a U-maze. In this test, puppies are placed within a closed U-shaped barrier so that they can see their handler and a bowl of food through a wire screen located at the apex of the maze. To exit the maze, they must turn away from their handler and pass around the back of the maze. The time to accomplish this is measured. Puppies are given 3 minutes to solve the maze before they are removed for that try. This process is repeated 5 times per session and puppies are tested in 2 different sessions one week apart. Results are summarized in Table 2.

TABLE 2
Mean times for U-maze (seconds).

| | Trial 1 | Trial 2 |
|---|---|---|
| Treatment A | 27.0 | 17.3 |
| Treatment B | 31.3 | 14.3 |

At 12 weeks of age problem solving and memory were tested in a long maze. In this test, puppies were placed in a long rectangular maze having 6 gates. A puppy was placed at one end of the maze while the handler stood at the far end and called it to come to a bowl of food. To reach the handler, the puppy had to find and pass through the open side of each gate. Each puppy ran the same pattern, but the pattern was alternated between successive puppies so that scent could not be used to solve the maze. The time to solve the maze and the number of errors were recorded for each run. Each session consisted of 3 runs through the maze.

Puppies were tested once a week for a total of 3 sessions. No differences between treatment groups were found for the first trial. On the second trial, the mean, median, and minimum run times were significantly lower (p<0.05) for the treatment group (B) puppies. The median and minimum number of errors were directionally (p<0.10) lower for the treatment B puppies. The DHA/AA treated dogs did perform better in the second running of the long maze. These findings suggest that memory was enhanced by DHA/AA supplementation. These data are summarized in Table 3.

TABLE 3
Values for Long Maze.

| | Trial 1 | Trial 1 | Trial 2 | Trial 2 |
|---|---|---|---|---|
| | Run time (sec) | errors | Run time (sec) | errors |
| Mean of | | | | |
| replicated runs | | | | |
| Treatment A | 74.8 | 4.2 | 55.3 | 4.2 |
| Treatment B | 94.2 | 4.3 | 43.7 | 2.8 |
| p-value A = B | 0.51 | 0.73 | 0.03 | 0.15 |
| Median of | | | | |
| replicated runs | | | | |
| Treatment A | 79.0 | 4.0 | 53.3 | 4.0 |
| Treatment B | 87.5 | 4.5 | 40.5 | 3.0 |
| p-value A = B | 0.56 | 0.79 | 0.03 | 0.07 |
| Minimum of | | | | |
| replicated runs | | | | |
| Treatment A | 52.5 | 3.0 | 37.5 | 2.0 |
| Treatment B | 57 | 3.0 | 25.0 | 1.0 |
| p-value A = B | 0.33 | 0.33 | 0.05 | 0.08 |

At 15 weeks of age, the puppies were tested for cue association in a T-maze. In this test, puppies watched as a bell was suspended and rung in front of one of two entrances each covered by a curtain. After the bell is rung, it is removed and the pup must wait 30 seconds before entering the chamber where the puppy can choose a side to enter. Previously, the pup was been shown that the side where the bell was rung leads to the exit of the maze and food, while the other side is a dead end. In the T-maze, trials consisted of 10 runs and all puppies were tested for 2 trials on successive weeks. Additionally, any puppies that had not made 7 of 10 correct choices after 2 trials were continued until 7 of 10 correct choices were made within a trial. Up to 4 trials in the T-maze were conducted. No differences between treatment groups were found for the first trial. On the second trial, mean run time was directionally (p<0.10) lower for the treatment A puppies. The corresponding error rate was numerically lower. Median run time also was numerically lower for the treatment A puppies. These data are summarized in Table 4.

TABLE 4
Values for T-Maze.

| | Trial 1 | Trial 2 | Trial 1 | Trial 2 |
|---|---|---|---|---|
| | run time | errors | run time | errors |
| Mean replicated | | | | |
| runs | | | | |
| Treatment A | 11.5 | 5 | 3.85 | 0.3 |
| Treatment B | 11.2 | 5 | 5.75 | 0.5 |
| P-value A = B | 0.85 | 0.95 | 0.85 | 0.12 |
| Median | | | | |
| replicated runs | | | | |
| Treatment A | 7 | | 2.75 | |
| Treatment B | 5 | | 3.50 | |
| p-value A = B | 0.7 | | 0.30 | |

For the long maze and T-maze, several responses were derived from the multiple runs. Mean, median and minimum run time and error rate were used as responses in the long maze. Mean run time and error rate and median run time were used as responses in the T-maze. The distribution of most responses showed significant deviation from a normal distribution. Differences between treatment groups for each response were tested by non-parametric analysis of variance performed on the ranks of the data after accounting for differences in the distribution of litters within treatment groups.

Biochemical tests. Blood samples were collected from all puppies at ages 8 and 16 weeks. Blood samples were centrifuged at 10,00×G and plasma removed. The red cells were washed 3 times with isotonic saline and then stored in vials. All tissue samples (milk, plasma and red blood cells) were placed in freeing vials and covered in nitrogen gas before storing at −70° C. until analysis.

The values for plasma fatty acid analysis for samples collected at 8 and 16 weeks are presented in are presented in Tables 5 and 6, respectively. Plasma DHA values were nearly 4-fold higher in treatment group B than in treatment group A dogs for samples taken at both 8 and 16 weeks of age. There were no significant differences in AA, LA, DPA or EPA values between treatment groups for either time period.

TABLE 5
Fatty acid analysis from blood plasma collected at 8 weeks of age
(Relative % of total fatty acids)

| | LA | AA | EPA | DPA | DHA |
|---|---|---|---|---|---|
| Treatment A | 24.6 | 16.77 | .18 | .356 | 1.25 |
| Treatment B | 22.8 | 17.23 | .33 | .325 | 4.78 |
| P-value A = B | .026 | .278 | <0.001 | .235 | <0.00001 |

TABLE 6
Fatty acid analysis from blood plasma collected at 16 weeks of age
(Relative % of total fatty acids)

| | LA | AA | EPA | DPA | DHA |
|---|---|---|---|---|---|
| Treatment A | 22.48 | 16.76 | 0.179 | 0.555 | 0.74 |
| Treatment B | 20.99 | 17.07 | 0.290 | 0.676 | 3.58 |
| P-value A = B | .005 | 0.313 | <0.0001 | 0.009 | <0.0001 |

The membrane fatty acid values obtained from RBC's collected at 16 weeks of age are presented in Table 7. As was found in with plasma fatty acid analysis, RBC membrane DHA values were significantly higher in treatment group B than in treatment group A dogs. The difference between treatment groups in RBC membrane DHA concentration was more than twice that observed in plasma samples.

TABLE 7
RBC membrane fatty acid content at 16 weeks of age
(Relative % of total fatty acids)

| | LA | AA | EPA | DPA | DHA |
|---|---|---|---|---|---|
| Treatment A | 11.617 | 21.316 | 0.139 | 0.42 | 0.346 |
| Treatment B | 10.812 | 22.735 | 0.226 | 0.344 | 2.65 |
| P-value A = B | 0.012 | 0.053 | <0.00001 | 0.004 | <0.00001 |

### REFERENCES

1. Bauer, J. E., Heinemann, K. M., Bigley, K. E., Lees, G. E., and Waldron, M. K. (2004) Maternal Diet Alpha-Linolenic Acid During Gestation and Lactation Does Not Increase Canine Milk Docosahexaenoic Acid, J Nutr 134, 2035S-2038S.
2. Bauer, J. E., Heinemann, K. M., Bigley, K. E., and Waldron, M. K. (2004) Enrichment of canine milk with n-3 LCPUFA is dose-dependent on gestation/lactation diet. ABSTRACT 6^{th} Congress of ISSFAL, Brighton, UK
3. Birch, E. E., Garfield, S., Hoffman, D. R., Uauy, R., and Birch, D. G. (2000) A randomized controlled trial of early dietary supply of long-chain polyunsaturated fatty acids and mental development in term infants. Dev. Med. Child Neurol. 42, 174-178.
4. Carlson, S. E., Werkman, S. H., Rhodes, P. G., and Tolley, E. A. (1993) Visual-acuity development in healthy preterm infants: Effect of marine-oil supplementation, Am. J. Clin. Nutr. 58, 35-42.
5. Carlson, S. E. (1996) Arachidonic acid status of human infants: influence of gestational age at birth and diets with very long chain n-3 and n-6 fatty acids. J. Nutr. 126(4 Suppl), 1092S-8.
6. Connor, W. E., Neuringer, M., and Lin, D. (1990) Dietary effects on brain fatty acid composition: the reversibility of n-3 fatty acid deficiency and turnover of docosahexaenoic acid in the brain, erythrocytes, and plasma of rhesus monkeys. J. Lipid Res. 31, 237-241.
7. Gibson, R. A., Neumann, M. A., and Makrides, M. (1997) Effect of increasing breast milk docosahexaenoic acid on plasma and erythrocyte phospholipid fatty acids and neural indices of exclusively breast fed infants. Eur. J. Clin. Nutr. 51, 578-82.
8. Gil, A., Ramirez, M., and Gil, M. (2003) Role of long-chain polyunsaturated fatty acids in infant nutrition, Eur. J. Clin. Nutr. 57, S31-57.
9. Green, P., and Yavin, E. (1996) Fatty acid composition of late embryonic and early postnatal rat brain. Lipids 31, S859-865.
10. Greiner, R. C., Winter, J., Nathanielsz, P. W., and Brenna, J. T. (1997) Brain Docosahexaenoate Accretion in Fetal Baboons: Bioequivalence of Dietary Alpha-Linolenic and Docosahexaenoic Acids, Pediatr Res 42, 826-834.
11. Greiner, R. S., Moriguchi, T., Hutton, A., Slotnick, B. M., and Salem, N. (1999) Rats with low levels of brain docosahexaenoic acid show impaired performance in olfactory-based and spatial learning tasks. Lipids 34 Suppl, S239-43.
12. Litman, B. J., Niu, S. L., Polozova, A., and Mitchell, D. C. (2001) The Role of Docosahexaenoic Acid Containing Phospholipids in Modulating G Protein-Coupled Signaling Pathways, J Molec Neurosci 16, 237-242.
13. Liu C. C., Carlson, S. E., Rhodes, P. G., and Rao, V. S. (1987) Meydrech Increase in plasma phospholipid docosahexaenoic and eicosapentaenoic acids as a reflection of their intake and mode of administration. Pediatr. Res. 22, 292-6.
14. Lothaller, M. A., and Widhalm, K. (1991) Are omega-3-fatty acids essential for newborn infants?(Sind omega-3-Fettsauren fur das Neugeborene essentiell?) Infusionstherapie 18, 280-282.
15. Martinez, M. (1992) Tissue Levels of Polyunsaturated Fatty Acids During Early Human Development, J Pediatr 120, S129-138.
16. Menard, C. R., Goodman, K. J., Corso, T. N., Brenna, J. T., and Cunnane, S. C. (1998) Recycling of Carbon Into Lipids Synthesized de Novo is a Quantitatively Important Pathway of Alpha-[U-13C]Linolenate Utilization in the Developing Rat Brain, J Neurochem 71, 2151-2158.
17. Neuringer, M., Conner, W. E., Van Paten, C., and Barstow, L. (1984) Dietary omega-3 fatty acid deficiency and visual loss in infant rhesus monkeys. J. Clin. Invest. 73, 272-76.
18. Pawlosky R. J., Denkins, Y., Ward, G. et al. (1997) Retinal and brain accretion of long-chain polyunsaturated fatty acids in developing felines: The effects of corn-based maternal diets. Am. J. Nutr. 665, 465-472.
19. Rotstein, N. P., Aveldano, M. I., and Politi, L. E. (1999) Essentiality of docosahexaenoic acid in retina photoreceptor cell development. Lipids 34, S15.
20. Sinclair, A. J. (1975) Long Chain Polyunsaturated Fatty Acids in the Mammalian Brain, Proc. Nutr. Soc. 34, 287-291.
21. Uauy, R., Hoffman, D. R., Mena, P., Llanos, A., and Birch, E. E. (2003) Term infant studies of DHA and ARA supplementation on neurodevelopment: results of randomized controlled trials, J. Pediatr. 143, 17-25.
22. Wainwright, P. E., Xing, H. C., Ward, G. R., Huang, Y. S./Bobik, E., Auestad, N., and Montalto, M. (1999) Water maze performance is unaffected in artificially reared rats fed diets supplemented with arachidonic acid and docosahexaenoic acid. J Nutr. 129, 1079-1089.
Willats, P. (2000) Long chain polyunsaturated fatty acids improve cognitive development. J. Fam. Health Care. 12, 5. Kelley, R. L., Lepine, A. J., Burr, J. R. (2004) Effect of dietary fish oil on puppy trainability. Proc. International Society for the Study of Fatty Acids and Lipids, Jun. 28-Jul. 1, 2004, Brighton, UK (Abst. 1-5).

The present invention is not limited to the embodiments described and exemplified above, but is capable of variation and modification within the scope of the appended claims.

What is claimed:

1. A composition comprising one or more long chain polyunsaturated fatty acids (LCPUFA), in an amount effective for improving cognitive function in an animal.

2. The composition of claim 1 wherein the composition is a pet food composition or a dietary supplement.

3. The composition of claim 1 wherein the LCPUFA include at least one of arachidonic acid, linoleic acid, eicosapentaenoic acid, docosapentaenoic acid, or docosahexaenoic acid.

4. The composition of claim 1, wherein the LCPUFA include at least one n-6 LCPUFA and at least one n-3 LCPUFA.

5. The composition of claim 1, wherein the LCPUFA are present in an amount of at least about 0.1% to about 10% by weight of the composition.

6. The composition of claim 1, wherein the LCPUFA are present in an amount of at least 0.4% to about 5.0% by weight of the composition.

7. The composition of claim 1, wherein the animal is a companion animal.

8. The composition of claim 7, wherein the companion animal is a dog or cat.

9. A method for enhancing cognitive function in an animal comprising administering to the animal one or more LCPUFA in an amount effective to enhance cognitive function in the animal.

10. The method of claim 9, wherein the LCPUFA are administered to the animal during gestation.

11. The method of claim 9, wherein the LCPUFA are administered to the animal during the period spanning parturition through about twelve weeks after parturition.

12. The method of claim 9, wherein the LCPUFA are administered to the animal during gestation and during the period spanning parturition through about twelve weeks after parturition.

13. The method of claim 9, wherein the LCPUFA are administered in a pet food composition or a dietary supplement.

14. The method of claim 9, wherein the LCPUFA are administered in milk from a lactating animal to which has been administered one or more LCPUFA.

15. The method of claim 9, wherein the LCPUFA are administered in a pet food composition or dietary supplement and in milk from a lactating animal to which has been administered one or more LCPUFA.

16. The method of claim 9, wherein the LCPUFA include one or more of arachidonic acid, eicosapentaenoic acid, docosapentaenoic acid, or docosahexaenoic acid.

17. The method of claim 9, wherein the LCPUFA include at least one n-6 LCPUFA and at least one n-3 LCPUFA.

18. The method of claim 9, wherein the animal is a companion animal.

19. The method of claim 18, wherein the companion animal is a dog or cat.

20. The method of claim 9, wherein the LCPUFA are administered to the animal on a daily basis.

21. The method of claim 9, wherein the LCPUFA are administered to the animal as part of a dietary regimen.

22. The method of claim 21, wherein the duration of the dietary regimen ranges from parturition to about 12 weeks of age.

23. The method of claim 9, wherein the LCPUFA administered to the animal comprise from about 0.1% to about 15% of the total daily caloric requirement of the animal.
